(19) United States
(12) Patent Application Publication
(10) Pub. No.: US 2008/0254108 A1
(43) Pub. Date: Oct. 16, 2008

(54) Formulations and Methods for Modulating Satiety

(76) Inventor: Mark Rosenberg, Del Ray Beach, FL (US)

Correspondence Address: MCDERMOTT WILL & EMERY LLP, 18191 VON KARMAN AVE., SUITE 500, IRVINE, CA, 92612-7108, US

(21) Appl. No.: 11/661,014

(22) PCT Filed: Aug. 23, 2005
(86) PCT No.: PCT/US2005/030108

§ 371 Date: Dec. 3, 2007

Related U.S. Application Data
(60) Provisional application No. 60/603,753, filed on Aug. 23, 2004.
(60) Provisional application No. 60/650,524, filed on Feb. 8, 2005.

(51) Int. Cl. A61K 38/08; A61K 38/02; A61K 38/07; A61K 9/127

(52) U.S. Cl. 424/450; 514/2; 514/16; 514/18

(57) ABSTRACT

This disclosure relates to formulations and methods of suppressing appetite and eliciting satiety (sense of being filled) in mammals through the oral administration of an effective amount of an appetite suppressing moiety.

### TECHNICAL FIELD OF THE INVENTION

The invention relates to the field of appetite management and suppression. More specifically, the invention relates to methods, compositions for modulating satiety and thus, to approaches useful to control weight, especially to manage excess weight and obesity.

### BACKGROUND OF THE INVENTION

Excess weight, ranging from being overweight to obesity and culminating in morbid obesity, is a major concern for industrialized nations because of the deleterious health effects overweight and obesity have been associated with. Overweight and obesity are diseases of excess energy stores in the form of fat. Overweight individuals have a Body Mass Index (BMI) of between 26 kg/m² and 30 kg/m² and obese individuals have a BMI of between 30 kg/m² and 40 kg/m². Moreover, morbid obesity (this term is synonymous with “clinically severe obesity”) correlates with a Body Mass Index (BMI) of at least 40 kg/m² or with being 100 pounds overweight.

The most obvious effect on health is an increased mortality rate directly related to weight increase (Lew et al., 1979, J. Chronic. Dis. 32:563). In a 12 year follow-up of 336,442 men and 419,060 women, it was found that the mortality rates for men 50% above average weight were increased approximately two fold. In the same weight group, the mortality was increased five fold for diabetics and four fold for those with digestive tract disease. In women, the mortality was also increased two fold, while in female diabetics, the mortality risk increased eight fold and three fold in those with digestive tract disease. It is clear that overweight people of both sexes, especially young overweight people, tend to die sooner than their lean contemporaries (Build and Blood Pressure Study, 1959, Chicago Society of Actuaries; Blair et al., 1966, Society of Actuaries 18:35; Stevens et al., 1998, N. Engl. J. Med. 338:1). While obesity, of itself, is a risk factor (see, Hubert et al., 1983, Circulation 67:968), most mortality and morbidity is associated with the co-morbid conditions.

Being overweight correlates with physical problems that are now well recognized. These conditions have been outlined in the 1985 National Institutes of Health Consensus Conference and include diabetes, gallstones, hypertension, heart disease (overweight individuals have an elevated heart disease risk as compared with healthy weight individuals, with a risk as high as three fold in obese patients), stroke, osteoporosis, and colon cancer making obesity a more substantial progenitor to disease than smoking, drinking, or poverty.

Lack of respect for the morbidly obese is also an issue of concern. A survey of severely obese individuals found that nearly eighty percent reported being treated disrespectfully by the medical profession (Maddox et al., 1968, J. Med. Ed. 44:214; Kurland et al., 1970, Psychiatric Opinion 7:20). There are widespread negative attitudes that the morbidly obese adult is weak-willed, ugly, awkward, self-indulgent and (in somefora, even as much as immoral). This intense prejudice cuts across age, sex, religion, race, and socioeconomic status. Numerous studies have documented the stigmatization of obese persons in most areas of social functioning. This can promote psychological distress and increase the risk of developing a psychological disorder. The morbidly obese patient is at risk for affective, anxiety and substance abuse disorders. The obese often consider their condition as a greater handicap than deafness, dyslexia or blindness (Rand et al., 1990, South Med. J. 83:1390; Rand et al., 1991, Int. J. Obes. 15:577).

Overweight and obesity have become a public health epidemic. According to the Centers for Disease Control and Prevention (“CDC”), the prevalence of overweight and obese individuals in the United States in 2000 was an estimated 64% of adults (˜120 million people). Of those, nearly half (˜59 million people) are obese. Moreover, the prevalence of overweight and obesity in children in the United States in 2000 was an estimated 15% of children aged 6 to 19. Unfortunately, the CDC also reports that the problem is getting worse and that in 2000 the cost of obesity in the United States was more than $117 billion (Overweight and Obesity: Frequently Asked Questions [online], June 2004 [retrieved on Jul. 05, 2004]; retrieved from the Internet: <URL: http://www.cdc.gov/nccdphp/dnpa/obesity/faq.htm#adults>).

Because of the enormous health impact in industrialized nations, especially the United States, there is a great need to control and maintain a healthy weight. Presently, weight control is attempted through a variety of approaches including increasing one's metabolic rate or decreasing one's appetite and thus, caloric intake. Methodologies often advocated to manage excess weight include exercise, diet, nutraceuticals, even medications or surgery (e.g., bariatric surgery), and often, a regimen combining these.

The many manifestations of diets and exercise routines have worked to some degree for some individuals but have ultimately failed the population as a whole. Diets are often difficult to maintain and may place the patient in danger if done incorrectly, leading to a “yo-yo” of weight gain and weight loss. For example, such a “yo-yo” effect places a strain on the patient's cardiovascular system. Moreover, the overall success rate of weight control after a year for such treatments is approximately a mere 5% (National Association to Advance Fat Acceptance (NAAFA); NAAFA Policy: Dieting and the Diet Industry [online], May 1993 [retrieved on Jul. 05, 2004]; retrieved from the Internet: <URL: http://www.naafa.org/documents/policies/dieting.html>).

Surgical treatments have met some measure of success but are invasive and in some cases fatal. Assessing the risks of surgical treatment of obesity involves operative, perioperative and long term complications. Morbidity in the early postoperative period (i.e., wound infections, dehiscence, leaks from staple breakdown, stomal stenosis, marginal ulcers, various pulmonary problems, and deep thrombophlebitis), may be as high as ten percent or more. Splenectomy is necessary in 0.30% of patients to control operative bleeding. In the late postoperative period, other problems may arise and may require reoperation. According to researchers, patients followed for 5 years after a gastric bypass had lost an average of 97 pounds. As a result, patients having the surgery resolved issues associated with diabetes and have been able to maintain a healthier body weight for longer than compared to dieters (Schauer et al., 2003, Ann. Surg. 238:467-485). Gastric bypass treatments, however, are only appropriate for those with a BMI of 40 kg/m² or greater (Yanovski et ail., 2002, N. Engl. J. Med. 346:591-602).

Another measure taken to treat obesity is prescription medication. Currently there are several medications on the market (i.e., Mazindol sold under the trademarks SANOREX and MAZANOR or, Phendimetrazine sold under the trademarks BONTRIL, PLEGINE, PRELU-2, and X-TROZINE) for short term use with obese patients. Unfortunately, the average weight loss is a relatively insignificant ranging from 5 to 22 pounds when compared to a target loss of 100 or more pounds. Some medications, like Orlistat and Sibutramiine (sold under the trademarks XENICAL and MERIDIA respectively), are indicated for long term use but may have considerable risks associated with their use. Combination therapies have also been employed with some success but often with serious consequences. For example, the appetite suppression cocktail fenfluramine and phentermine (“fen/phen”) has been withdrawn from the market due to fatalities attributed to fenfluramine. In addition, many of these medications are controlled substances and may have other serious side-effects that include the potential for dependence and the development of tolerance to the medication (National Institutes of Health (NIH), National Institute of Diabetes and Digestive and Kidney Diseases (NIDDK). Prescription Medications for the Treatment of Obesity [online], June 2003 [retrieved on Jul. 05, 2004]. Retrieved from the Internet: <URL: http://www.niddk.nih.gov/health/nutrit/pubs/presmeds.htm#single>).

While each of these lines of attack may be successful in some individuals, there remains a considerable portion of refractory patients in need of new alternative methodologies.

Considerable attention has been given to neuromodulators and neurotransmitters postulated to play a role as peripheral negative feedback signals in feeding behavior. Since the early 70's the hypothesis that satiety elicited by food in the intestine is mediated by one or more gut peptides released by preabsorptive food stimuli which act as peripheral negative feedback signals to stop feeding behavior has received ample support in the literature (see e.g., Gibbs et al., 1972, Fed. Proc. 31:397). A substantial body of evidence has showed that peripherally administered cholecystokinin (“CCK”) or caerulein, a decapeptide closely related to CCK, produce the behavioral sequence in various systems including murine as well as in primate characteristic of postprandial satiety, i.e., the animals behaved as if they had received food (see e.g., Antin et al., 1975, J. Comp. Physiol. Psychol. 89:784, and Stacher et al., 1982, Peptides 3:607).

Although the consensus is that peripherally administered CCK as well as caerulein is able to reduce food intake in man, the mechanism by which this effect is brought about have not definitively been elucidated. There is good evidence, however, that the site of action is on an abdominal organ innervated by gastric vagal branches and relayed to the brain by afferent vagal fibers (reference omitted). It has been postulated that gastric vagal afferents could be activated by CCK by responding to the effect of the peptide on gastric smooth muscle and to the activation of vagal receptors for muscle tension and stretch. CCK relaxes the proximal stomach and contracts the pyloric region. Both effects, which result in a deceleration of gastric emptying lead to gastric distension when more food is ingested decreasing overall intake.

Notably, any enthusiasm raised by the many studies showing CCK and caerulein effectiveness in modulating feeding behavior has been quashed by sub-therapeutic oral bioavailability. It has been discovered that CCK and caerulin, like many other peptides cannot be effectively administered by the oral route because of intestinal metabolism and poor systemic absorption from the gastrointestinal tract. To be effective, CCK and caerulein would need to be administered via intravenous or intramuscular routes, requiring intervention by a physician or other health care professional, entailing considerable discomfort and potential local trauma to the patient. Due to these considerable practical considerations, neither one of these active compounds has been exploited to manage excess weight and obesity despite their impressive potential.

None of the published studies provides any regimen for implementing the effective oral administration of either CCK or caerulein, e.g., indicating the respective dosage ranges, mode of administration for specific target drugs and bioavailability-enhancing agents or demonstrating agents are best suited for promoting oral absorption of each target drug. Methods disclosed in the art for increasing absorption of CCK or caerulein (that, to date, have been successfully administered only parenterally) generally focus on the use of permeation, solubility enhancers as promoting agents, or on co-administration by intraluminal perfusion in the small intestine or by the intravenous route of enzyme inhibitors (e.g., Su et al., 2002, Biochem. and Biophys. Res. Comm. 292:632). Notably, the art fails to identify suitable formulations or specific treatment regimens and schedules which would render the target agents therapeutically effective upon oral administration.

J Thus, a safe yet effective method for increasing the systemic availability upon oral administration of drugs that are currently administered only parenterally because they are not absorbed sufficiently or consistently when administered by the oral route is required and has not yet been provided.

### SUMMARY OF THE INVENTION

The invention is based on the unexpected and surprising discovery that certain appetite suppressing (satiety) moieties associated in the literature with sub-therapeutic oral bioavailability may be orally bioavailable when formulated as described herein. The invention further provides methods of administering such formulations to enhance bioavailability. The invention also provides methods of using such formulations in a patient to treat various conditions. As exemplified, the pharmaceutical formulations and methods according to the invention are shown to modulate/induce satiety and reduce feeding as evidenced by a marked weight loss in treated patients as compared to those untreated.

Accordingly, it is an object of the present invention to provide pharmaceutical formulations suitable for oral administration. Another object of the present invention is to reduce the caloric intake in treated patients by the oral administration of the formulations according to the invention. Yet another object of the invention is to cause weight loss resulting from a reduced caloric intake. In some patients, an object of the invention is to manage weight (and in some instances to preserve weight) by modulating appetite, and thus caloric intake, by the oral administration of appetite suppressing (satiety) peptides formulated according to the invention.

Another aspect of the invention is to provide formulations and methods to provide better glycemic control for Type II diabetics. An object of the invention is thus, to help Type II diabetics to enhance and extend satiety. Another object is thus to reduce caloric intake in Type II diabetics. In another aspect, the invention provides formulations and methods to increase satiety in bulimics who have a defect in their normal CCK release mechanism.

### DESCRIPTION OF THE INVENTION

The invention relates to the idea that certain oral formulations of appetite suppressing (satiety) moieties may play an important role in weight-control. Specifically, it is shown that oral administration of appetite suppressing peptides and related moieties increases appetite suppression, thereby leading to increased feelings of satiety, which, in turn lower food consumption and ultimately results in weight-loss.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

In the specification and the appended claims, the singular forms include plural referents unless the context clearly dictates otherwise.

As used in this specification, the singular forms “a,” “an” and “the” specifically also encompass the plural forms of the terms to which they refer, unless the content clearly dictates otherwise.

As used herein, unless specifically indicated otherwise, the word “or” is used in the “inclusive” sense of “and/or” and not the “exclusive” sense of “either/or.”

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

Accordingly, in an aspect the invention sets forth formulations and methods suitable for oral administration of appetite suppressing (satiety) moieties previously believed to be poorly available, if not unavailable, upon oral administration. As shown hereinafter, the formulations of the invention are useful to modulate/induce satiety and reduce feeding as attested and measured by a body-weight reduction in treated patients as compared with untreated patients. Throughout this application, the expressions “reduce caloric intake,” “reduce food intake” or “reduce feeding” are used interchangeably to denote a reduction in caloric intake regardless as to whether solid or liquid food or other form of nutrition is involved.

The compositions and methods of the present invention are intended for use with any patient, preferably a mammal, which may experience the benefits of the invention. Foremost among such mammals are humans, although the invention is not intended to be so limited, and is applicable to veterinary uses. Thus, in accordance with the invention, “patient”, “mammals” or “mammal in need” include humans as well as non-human mammals, particularly domesticated animals including, without limitation, cats, dogs, and horses.

Any suitable materials and/or methods known to those of skill can be utilized in carrying out the present invention. However, preferred materials and methods are described. Materials, reagents and the like to which reference is made in the following description and examples are obtainable from commercial sources, unless otherwise noted.

An aspect of the invention provides a pharmaceutical formulation for oral administration to a patient to modulate/induce satiety and reduce feeding comprising an appetite suppressing peptide, and a chelating agent, wherein the formulation is encased in an enteric coating or capsule.

The formulations of the compositions according to the invention are prepared in a pharmaceutically acceptable vehicle with any of the well known pharmaceutically acceptable carriers, including diluents and excipients (see, Remington's Pharmaceutical Sciences, 18^{th} Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compounds of the invention.

In particular, permeation enhancers are contemplated. These substances can operate by increasing either paracellular or transcellular transport systems. An increase in paracellular transport can be achieved by opening the tight junctions of the cells; an increase in transcellular transport can be achieved by increasing the fluidity of the cell membrane. Paracellular permeation enhancers include a variety of moiety known in the art (see e.g., (see, Remington's Pharmaceutical Sciences, 1990, supra, and Remington: The Science and Practice of Pharmacy, 1995, stipra). Representative, non-limiting examples of such permeation enhancers include for example calcium chelators, bile salts (such as sodium cholate), fatty acids, and sterylglucoside. Both short and medium chain fatty acids have shown to enhance the uptake of minerals (Fe and Ca), as well as other substances, via the augmentation of paracellular transport.

In certain embodiments, the compositions of the invention may be delivered in conjunction with a fatty acid (e.g., oleate, palmitate, stearate, sodium caprate, or conjugated linoleic acid) in an enteric-coated capsule, with the goal of increasing bioavailability via increased paracellular transport. The propensity of ligand-modified liposomes to carry drugs and genes to desirable sites has been extensively examined and current reports in the literature show considerable progress in this field. One of skill in the art will recognize that a variety of possible moieties may be used for the purpose. Thus, for example, sterylglucoside (SG) is a relatively recent absorption-enhancer of peptide drugs across nasal and intestinal mucosae. Physico-chemical properties and biodistribution of liposomes incorporating SG have revealed that SG particles aids intestinal drug delivery and increases bioavailability of peptide drugs after nasal and intestinal administration.

Bioavailability of the compositions may be further enhanced by coating liposomes with polyethylene glycol and related moieties known to interact with the mucus layer of the GI tract, to modulate transit rate. Both bile salts and fatty acids are individually, permeation enhancers. Studies have been performed on both GI and nasal mucosa, revealing that the bile salt, “sodium glycocholate, (NaGC)” when added to a fatty acid such as linoleic acid, to form mixed micelles, enhances the absorption of a peptide greater than that seen with the use of the bile salt alone.

Compositions of the invention may be delivered orally in any of the carrier vehicles described herein, in conjunction with a bile salt, such as NAGC, and a fatty acid, such as linoleic acid, with the intent of improving bioavailability via permeation enhancement. The mucus layer barrier of the intestinal epithelium is often underestimated and can be a formidable obstacle to the absorption of peptide drugs. Detergents, sulfhydryl compounds, and mucolytic enzymes are reported to display mucolytic activity, thereby improving peptide absorption. Sulfhydryl compounds display more potent hydrolytic activity than detergents by cleaving disulfide bonds which connect mucus glycoproteins with each other. A well-established sulfhydryl compound of high mucolytic activity is N-acetylcysteine, which is used as an expectorant in various pharmaceutical preparations. In vivo studies focusing on the influence of the mucus gel layer on intestinal permeability, demonstrated a significantly higher uptake of FITc-dextran 70,000 in rats due to the co-administration of N-acetylcysteine. Another potent sulfhydryl compound is dithiothreitol. In ileum and proximal colon, this agent increased the absorption and biliary recovery of a tripeptide four-fold and 70-fold over control rats respectively.

Compositions of the invention may be delivered to the small intestine in conjunction with a sulfhydryl compound, such as for example N-acetylcysteine or dithiothreitol.

Permeation enhancers useful according to the invention include the high molecular mass polymers such as chitosan and polyacrylates. Their mucoadhesive properties allow them to remain concentrated at the area of drug absorption. In general, these polymers are divided into cationic and anionic polymers. Representative for cationic polymers is the widely used chitosan. The permeation enhancing effect of this polymer could be demonstrated via various studies on Caco-2 monolayers and in vivo rat models. The underlying mechanism of opening of tight junctions by chitosan was attributed to the interaction of the positively charged amino groups with the negatively charged sialic groups of membrane-bound glycoproteins. Furthermore, anionic polymers such as polycarbophil or carboxymethylcellulose also demonstrated permeation-enhancing properties. In contrast to the direct interaction of chitosan to the mucosal surface, these two polymers were shown to express a high calcium-binding ability. The depletion of calcium ions from the extracellular cell medium has been shown to increase the permeation of sodium-fluorescein, bacitracin, a vasopressin analogue, and insulin. Parallel measurement of the transepithelial electrical resistance (TEER) demonstrated a decrease in TEER indicating the opening of the tight junctions.

Chitosan derivatives are not soluble at pH above 6.5. In order to overcome this problem, N-trimethylation of chitosan chloride was tested and found to increase the solubility at higher pH. The use of this new trimethylated chitosan in vivo on rats was shown to significantly improve the absorption of octreotide after intrajejunal administration. Another chemical modification is the mono-N-carboxymethylation of chitosan. This resulted in an improved permeation of low molecular mass heparin in vitro and in vivo. Accordingly, such derivatives suitable at the pH of interest are contemplated.

Other promising types of chemically-modified polymers are thiolated polymers, or so called, “thiomers.” Due to the immobilization of free sulfhydryl groups onto various well-established polymeric excipients their permeation-enhancing effect on hydrophilic compounds such as sodium fluoresceine, rhodamine 123, bacitracin, insulin, or FITC-labeled HGH can be strongly improved. In addition, thiomers exhibit improved mucoadhesive properties, which allow it to remain concentrated at the area of drug absorption. Recently, the underlying mechanism of permeation enhancement by thiomers was shown to depend on the inhibition of protein tyrosine phosphatase (PTP). This results in a higher extent of phosphorylated tyrosine groups on the two loops of the membrane spanning protein Occludin, leading to the opening of the tight junctions.

Meanwhile, the high efficacy of the thiomers to enhance mucosal uptake has been shown in various in vivo studies. Caliceti et al., for instance, gained a pharmacological efficacy of 7% of orally applied PEG-ylated insulin in diabetic mice by incorporating the peptide in a minitablet based on poly(acrylic acid)-cysteine conjugate containing 2% of glutathione (see Caliceti et al., 2000, Pharm. Res. 17 12:1468-1474). Similarly, salmon calcitonin was also made more bioavailable; minitablets were again used, based on a thiolated chitosan with the addition of glutathione.

It is hereby proposed that compositions of the invention may be delivered orally (e.g., in a tablet), based on a chitosan derivative as mentioned above, that is soluble above pH 6.5, such as a thiolated chitosan, with and without the addition of glutathione. In many cases, the type of formulation itself influences the peptide drug absorption. Formulations such as nanoparticles and liposomes are reported to improve mucosal peptide drug absorption. Nanoparticles offer the advantage of protecting incorporated peptides from degradation. They can cross over the mucosal membrane either through Peyer's patch and/or the paracellular route. After having reached the systemic circulation, the particles are biodegraded releasing the incorporated peptide drug.

Compositions of the invention may be delivered (e.g., orally) in a liposomal or nanoparticle carrier. Mucoadhesive delivery systems are able to adhere on the mucus gel layer covering mucosal membranes, allowing for a prolonged stay of the peptide at the absorption site. Mucoadhesive strength of polymers is based on non-covalent bonds such as hydrogen bonding and ionic interactions or covalent bonds such as the formation of disulfide bonds with the mucus layer. These polymers adhere on mucosal surfaces. Polymers displaying high mucoadhesive properties are polyacrylates and chitosans. Their mucoadhesive properties can even be improved by the immobilization of thiol groups. Particles and liposomes can be coated with mucoadhesive polymers, or the mucoadhesive polymer can directly be used in the form of matrix tablets, microparticles, or nanoparticles.

“Appetite suppressing moieties”, interchangeably also referred to as “satiety moieties” are compounds known in the literature to reduce an individual's appetite, resulting in a reduced caloric intake and ultimately quantifiable by a detectable body-weight loss. Appetite suppressing moieties include naturally occurring peptides as well as synthetic peptides (see e.g., EP226217 and EP268297), peptidomimetics or other moiety containing a peptide bond.

One such appetite suppressing moiety is cholecystokinin and its derivatives, analogs, variants, as well as fragments thereof preserving appetite suppressing properties, pseudopeptides and peptidomimetics. Fragments and derivatives of CCK of particular interest include without limitation cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7, and cholecystokinin-4 (CCK-4). The sulfated form of CCK-8 has a high affinity for the CCK_{A} receptors, while the non-sulfated form of CCK-8, as well as CCK-4, gastrin, and pentagastrin (CCK-5) have a 10,000 fold lower affinity for these receptors (see, de Montigny, 1989, Arch. Gen. Psychiatry 46(6):511. The CCK_{B} receptors exhibit a high affinity and selectivity for CCK-4, gastrin, pentagastrin (CCK-5), and the non-sulfated CCK-8. Sulfated CCK-8 has a slightly lower or same affinity for CCK_{B} receptors (see, de Montigny, 1989, Arch. Gen. Psychiatry 46(6):511; Bradwejn et al., 1992b, Am. J. Psychiatry 149:962. Thus, sulfated CCK-8 is preferred in certain embodiments. Other appetite suppressing moieties contemplated include any CCK_{A} agonist having appetite suppressing properties, caerulein, Bombesin, and all other fragments of CCK containing at least the four C-terminal amino acids (Trp-Met-Asp-Phe-NH₂) (see, Abhiram, 2004, Endocrinology 145:2613).

CCK was identified in 1928 from preparations of intestinal extracts by its ability to stimulate gallbladder contraction. Other biological actions of CCK have since been reported, including stimulation of pancreatic secretion, delayed gastric emptying, stimulation of intestinal motility and stimulation of insulin secretion (see, Lieverse et al., 1994, Ann. N.Y. Acad. Sci., 713:268). The actions of CCK, also reportedly include effects on cardiovascular function, respiratory function, neurotoxicity and seizures, cancer cell proliferation, analgesia, sleep, sexual and reproductive behaviors, memory, anxiety and dopamine-mediated behaviors (Crawley and Corwin, 1994, Peptides 15:731). Other reported effects of CCK include stimulation of pancreatic growth, stimulation of gallbladder contraction, inhibition of gastric acid secretion, pancreatic polypeptide release and a contractile component of peristalsis. Additional reported effects of CCK include vasodilation (Walsh, “Gastrointestinal Hormnones,” In Physiology of the Gastrointestinal Tract (3d ed. 1994; Raven Press, New York)).

It has been reported that injections of combinations of glucagon, CCK and bombesin potentiated the inhibition of intake of condensed milk test meals in nondeprived rats over the inhibitions observed with individual compounds (Hinton et al., 1986, Brain Res. Bull. 17:615). It has also been reported that glucagon and CCK synergistically inhibit sham feeding in rats (LeSauter and Geary, Am. J. Physiol., 1987, 253:R217; Smith and Gibbs, 1994, Annals N.Y. Acad. Sci. 713:236). It has also been suggested that estradiol and CCK can have a synergistic effect on satiety (Dulawa et al., 1994, Peptides 15:913; Smith and Gibbs, supra). Experimental manipulations of exogenous and endogenous CCK and estradiol have produced converging evidence that estradiol cyclically increases the activity of the CCK satiation-signaling pathway so that meal size and food intake decrease during the ovulatory or estrous phase in animals (Geary, 2001, Peptides 22(8):1251). It is common-place for women who begin oral administration of estrogen (hormione replacement therapy or birth control) to gain weight. This occurs through several mechanisms including water retention; increased production of sex hormone binding globulin which thereby decreases bio-available testosterone; decrease in the production of IGF1. Thus, if oral estrogen replacement were given concurrently with an orally bio-available form of CCK, as proposed herein, it is likely that the usual weight gain would not occur. This would certainly be advantageous to women prone to obesity. It has also been proposed that signals arising from the small intestine in response to nutrients therein may interact synergistically with CCK to reduce food intake (Cox, 1990, Behav. Brain Res. 38:35). Additionally, it has been reported that CCK induces satiety in several species. For example, it has been reported that feeding depression was caused by CCK injected intraperitoneally in rats, intraarterially in pigs, intravenously in cats and pigs, into the cerebral ventricles in monkeys, rats, dogs and sheep, and intravenously in obese and nonobese humans (see e.g., Lieverse et al., stipra). Studies from several laboratories have reportedly confirmed the behavioral specificity of low doses of CCK on inhibition in feeding, by comparing responding for food to responding for nonfood reinforcers in both monkeys and rats and by showing that CCK elicits the sequence of behaviors normally observed after meal ingestion (i.e., the postprandial satiety sequence). Additionally, comparison of behavior after CCK to behavior after food ingestion, alone or in combination with CCK has reportedly revealed behavioral similarities between CCK and food ingestion (see e.g., Crawley and Corwin, supra). It has also been reported that CCK in physiological plasma concentrations inhibits food intake and increases satiety in both lean and obese humans (Lieverse et al., supra).

CCK was characterized in 1966 as a 33-amino acid peptide (Crawley and Corwin, supra). Species-specific molecular variants of the amino acid sequence of CCK have been identified. The 33-amino acid sequence and a truncated peptide, its 8-amino acid C-terminal sequence (CCK-8) have been reportedly identified in pig, rat, chicken, chinchilla, dog and humans. A 39-amino acid sequence was reportedly found in pig, dog and guinea pig. A 58-amino acid sequence was reported to have been found in cat, dog and humans. Frog and turtle reportedly show 47-amino acid sequences homologous to both CCK and gastrin. Very fresh human intestine has been reported to contain small amounts of an even larger molecule, termed CCK-83. In the rat, a principal intermediate form has been reportedly identified, and is termed CCK-22 (Physiology of the Gastrointestinal Tract, 3d Ed., Walsh, 1994; Raven Press, New York, N.Y. 1994).

A nonsulfated CCK-8 and a tetrapeptide (termed CCK-4 (CCK30-33)) have been reported in rat brain. The C-terminal penta peptide (termed CCK-4 (CCK 29-33)) conserves the structural homology of CCK, and homology with the neuropeptide, gastrin. The C-terminal sulfated octapeptide sequence, CCK-8, Asp-Tyr(SO₃H)-Met-Gly-Trp-Met-Asp-Phe-NH₂, is reportedly relatively conserved across species. Cloning and sequence analysis of a cDNA encoding preprocholecystokinin from rat thyroid carcinoma, porcine brain, and porcine intestine reportedly revealed 345 nucleotides coding for a precursor to CCK, which is 115 amino acids and contains all of the CCK sequences previously reported to have been isolated (see, Crawley and Corwin, supra).

CCK is said to be distributed throughout the central nervous system and in endocrine cells and enteric nerves of the upper small intestine. CCK agonists include CCK itself (also referred to as CCK-33), CCK-8 (CCK26-33), non-sulfated CCK-8, pentagastrin (CCK-5 or CCK(29-33)), and the tetrapeptide, CCK-4 (CCK30-33). At the pancreatic CCK receptor, CCK-8 reportedly displaced binding with a 1000-5000 greater potency than unsulfated CCK-8 or CCK-4, and CCK-8 has been reported to be approximately 1000-fold more potent than unsulfated CCK-8 or CCK-4 in stimulating pancreatic amylase secretion (see, Crawley and Corwin, supra). In homogenates from the cerebral cortex, CCK receptor binding was said to be displaced by unsulfated CCK-8 and by CCK-4 at concentrations that were equimolar, 10-fold or 100-fold greater than sulfated CCK-8.

Receptors for CCK have been reportedly identified in a variety of tissues, and two primary subtypes have been described: type A receptors and type B receptors. Type A receptors have been reported in peripheral tissues including pancreas, gallbladder, pyloric sphincter and afferent vagal fibers, and in discrete areas of the brain. The type A receptor subtype (CCK_{A}) has been reported to be selective for the sulfated octapeptide. Accordingly, in certain embodiments of the invention, the CCK fragment includes at least one sulfation group. CCK_{A} agonists also include A-71623 and A-708874, which were developed based on the structure of CCK-4. Members of another series of CCK_{A} agonists, which includes JMV-180, are reportedly active in stimulating pancreatic amylase release and inhibiting feeding (Crawley and Corwin, supra). Examples of non-peptide CCK_{A} agonists are L-364718 and FPL 15849KF (Crawley and Corwin, supra and Morley et al., 1994, Am. J. Physiol. 267:R178). Accordingly, substances which function as Type-A receptor-selective CCK agonists which may serve as anorectic agents are contemplated appetite suppressing moieties. These may include, without limitation, cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7, cholecystokinin-4 (CCK-4), caerulein, Bombesin, and all other fragments of CCK containing at least the four C-terminal amino acids (Trp-Met-Asp-Phe-NH₂).

J “Caerulein” refers to a specific decapeptide obtained from the skin of hila caerulea, an Australian amphibian. Caerulein is similar in action and composition to cholecystokinin. It stimulates gastric, biliary, and pancreatic secretion and certain smooth muscle (for a comprehensive review see e.g., Stacher et al., 1982, Peptides 3:607; Reidelberger et al., 1989, Am. J. Physiol. Regul. Integr. Comp. Physiol. 256:R1148; Anika, 1982, European J. Pharm. 85:195-199.).

A wide variety of medicaments, bioactive active substances and pharmaceutical compositions may be included in the formulations/dosage forms of the present invention to further enhance their therapeutic effects or to otherwise increase their benefit. Examples of useful active drugs include agents that act as agonists or antagonists to transmitters acting in the brain to increase satiety, including e.g., epinephrine antagonists, opiate antagonists, pancreatic polypeptide blockers, GABA agonists, serotonin agonists, calcitonin agonists, corticotropin-releasing factor agonists, neurotensin agonists; or to decrease hunger, including e.g., dopamine agonists, pancreatic polypeptide blockers, norepinephrine agonists, anesthetics, glucagon agonists, POMC, CART, urocortin, thyrotropin-releasing hormone, GLP-1, Galanin-like peptide-1, peptideY-Y, ciliary neurotrophic factor, brain-derived neural factor, insulin, IGF-1, IGF-11, leptin, neuropeptide K, calcitonin-gene related peptide, prolactin-releasing peptide, neuromedin and neuropeptide B, somatostatin, oxytocin, bombesin, motilin, enterostatin, anorectin, amylin, and interleukin 1 (Abharim, supra).

Without wishing to limit the scope of the invention or to be bound by any one mechanism, it is postulated that the poor bioavailability of these compounds documented in the literature is due to a combination of enzyme-specific degradation (e.g., specific peptidases) and to hydrolysis at acidic pHs. The inventor has devised a formulation approach combining the addition of a chelating agent with encasing in an enteric coating or capsule to protect the peptides from both enzymatic digestion as well as from hydrolysis.

The formulations according to the invention include, in addition to at least an appetite suppressing moiety (e.g., CCK or caerulein), a chelating agent. One skilled in the art will appreciate thus, that chelating agents are moieties capable of sequestering ions (which are cofactors participating in a variety of biochemical reactions) and thus, may impair the activity of many enzymes. Chemically chelators are organic chemicals that form two or more bonds with a metal ion forming a heterocyclic ring (e.g., porphyrin ring) with the metal atom as part of the ring.

Chelating agents are well known in the art. Non-limiting representative examples of chelating agents within the scope of the invention include ethylenediamine-N,N,N′,N′-tetraacetic acid (EDTA); the disodium, trisodium, tetrasodium, dipotassium, tripotassium, dilithium and diammonium salts of EDTA; the barium, calcium, cobalt, copper, dysprosium, europium, iron, indium, lanthanum, magnesium, manganese, nickel, samarium, strontium, and zinc chelates of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-te-traacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephos-phoric acid); 7,19,30-trioxa-1,4,10,13,16,22,27,33-octaazabicyclo[11,11,11]pentatriacontane hexahydrobromide; and triethylenetetramine-N,-N,N′,N″,N′″,N′″-hexaacetic acid, citric acid, and phosphoric acid. The calcium salt of EDTA is exemplified herein. One of skill in the art will appreciate that the amount of chelating agent included in the formulations according to the invention is significant to the stability of the formulation and ultimately to the overall bioavailability of the appetite suppressing moieties of the invention. In the embodiments of the invention, the chelating agent is present in an amount from about 25 mg to about 400 mg, or from about 100 mg to about 300 mg. In the illustrative embodiments exemplified herein 200 mg of calcium EDTA were included.

Additionally, formulations according to the invention may include specific enzyme inhibitors (e.g., any substrate that blocks the natural activity of a given enzyme) such as known peptidases inhibitors (e.g., thiorphan-a metalloendopeptidease inhibitor, amastatin-a competitive inhibitor of aminopeptidases, kelatophan, and neuropeptidases (e.g., endopeptidases (such as for example neurolysin and nephrilysin), aminopeptidases (e.g., proglutamyl aminopeptidase II, aminopeptidases N, A, B, and P), dipeptidases (e.g., NAALA dipeptidase), or carboxypeptidases (e.g., angiotensin converting enzyme homolog (ACEH), carboxypeptidases H, N, or P)).

Conveniently the formulations according to the invention are in the form of a tablet coated with a conventional enteric coating. Alternatively the formulations according to the invention may be presented in the form of a variety of oral dosage forms such as a capsule, the shell of which is made from enteric material or is coated with an enteric material.

In the context of this application it will be understood that the term enteric coating or material refers to a coating or material that will pass through the stomach essentially intact but will rapidly disintegrate in the small intestine to release the active drug substance. The enteric coating solution used consists of the following: cellulose acetate phthalate (“CAP”), ammonium hydroxide (27-31%), triacetin USP, ethyl alcohol (190 proof USP), methylene blue 1% solution, purified water. USP CAP is a polymer that has been used for several decades in the pharmaceutical industry for enterically coating individual dosage forms (e.g., tablets and capsules). CAP is not soluble in water at a pH of less than 5.8. The coating applied to our capsules will begin to break down upon being released into the duodenum and will be completely broken down by the time it reaches the ileum.

One of skill in the art, will appreciate that CAP is but one of many available enteric coating materials and that any other enteric coating material may be used according to the invention. An enteric coating according the invention is one which promotes dissolution of the dosage form primarily at a site outside the stomach. In some embodiments, the enteric coating of the invention promotes dissolution/breakdown of the dosage form to occur at a pH of approximately at least 6.0. In some instances, the coating is selected to promote dissolution at a pH of from about 6.0 to about 8.0 preferably favoring dissolution in the proximity of the ileum (i.e., at a pH of approximately 8.0).

The oral dosage forms (e.g., tablets and capsules) may also contain conventional excipients such as binding agents, (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc polyethylene glycol or silica), disintegrants (for example, potato starch or sodium starch glycollate) or wetting agents, such as sodium lauryl sulphate. In a preferred method, it is helpful to fill the unused space in the capsule with sufficient microcrystalline cellulose, so that there is minimal air (O₂) available in the capsule to oxidize and therefore degrade the CCK-8. To further prevent oxidation and ensure the stability of the CCK-8, it is envisioned that the capsule be prepared under a nitrogen hood. The enteric coatings may be applied to the tablets and/or capsules according to methods well-known in the art.

It will also be appreciated by those skilled in the art that the compounds of the present invention may also be utilized in the form of a pharmaceutically acceptable salt or solvate thereof. The physiologically acceptable salts of the appetite suppressant moieties of the invention include conventional salts formed from pharmaceutically acceptable inorganic or organic acids as well as quaternary ammonium acid addition salts. More specific examples of suitable salts include hydrochloric, hydrobromic, sulphuric, phosphoric, nitric, perchloric, fumaric, acetic, propionic, succinic, glycolic, formic, lactic, maleic, tartaric, citric, pamoic, malonic, hydroxymaleic, phenylacetic, glutamic, benzoic, salicylic, fumaric, toluenesulphonic, methanesulphonic, naphthalene-2-sulphonic, benzenesulphonic and the like. Other acids such as oxalic, while not in themselves pharmaceutically acceptable, may be useful in the preparation of salts useful as intermediates in obtaining the compounds of the invention and their pharmaceutically acceptable salts. References hereinafter to an appetite suppressing moiety for use in the invention include pharmaceutically acceptable salts and solvates.

Suitable enteric coatings for use in the invention will be these coatings known to those skilled in the art. Such coatings include without limitation, cellulose acetate phthalate, polyvinyl acetate phthalate, shellac, styrene maleic acid copolymers, methyacrylic acid copolymers (e.g., those marketed under the trademark EUDRAGIT) and hydroxypropyl methyl cellulose phthalate. The said coatings may also contain art known plasticizers and/or dye(s).

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001).

Another aspect of the invention provides a method to modulate/induce satiety and reduce feeding comprising the step of orally administrating to a patient a pharmaceutical formulation comprising an appetite suppressing peptide, and a chelating agent, wherein the formulation is encased in an enteric coating or capsule.

For use by the physician, the compositions will be provided in unit dosage form containing an amount of a compound of the invention (with or without another feeding suppressing agent) which will be effective in one or multiple doses to control appetite at the selected level.

Therapeutically effective amounts of an appetite modulator according to the invention, (e.g., CCK or caerulein), for use in reducing appetite and/or suppressing food intake and in conditions in which food intake is beneficially reduced are those treatments at dosages effective to achieve the therapeutic result sought. Furthermore, one of skill will appreciate that the therapeutically effective amount of the compound of the invention may be lowered or increased by fine timing and/or by administering more than one compound of the invention, or by administering a compound of the invention with another compound. The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

Such dosages of each of CCK are between about 0.1 μg/day and about 10 μg/day, preferably between about 0.1 μg/day and about 5 μg/day, administered in a single dose or in multiple doses. Such dosages of caerulein are between about 0.05 μg/day and about 5 μg/day, or between about 0.1 μg/day and about 4 μg/day, and between about 0.1 μg/day and 2.5 μg/day administered in a single dose or in multiple doses.

Generally, in suppressing appetite, the compounds of this invention may be administered to patients in need of such treatment in dosage ranges similar to those given above, however, the compounds may be administered more frequently, for example, one, two, or three times a day.

As mentioned above, the formulations of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc. Alternately, administration of a composition of all of the aspects of the present invention may be effected by liquid solutions, suspensions or elixirs, powders, lozenges, micronized particles and osmotic delivery systems.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated to provide a slow or controlled release of the active ingredient therein.

Dosage forms according to the invention may contain from about 0.1 to 10 μg of a appetite suppressing peptide and from about 0.1 to 500 mg of a chelating agent. Non-limiting example formulations include at least 2.4 μg of a appetite suppressing peptide such as for example CCK, from about 25 mg to about 400 mg of a chelating agent, such as for example calcium EDTA. An exemplified dosage form according to the invention includes, 4 μg of CCK, and 200 mg of calcium EDTA. Pharmaceutically acceptable excipients such as, for example, carboxymethylcellulose sodium or ethylcellulose, may incorporated into the dosage forms, if desired (see, Remington's Pharmaceutical Sciences, 18^{th} Ed., Gennaro, Mack Publishing Co., Easton, Pa. 1990 and Remington: The Science and Practice of Pharmacy, Lippincott, Williams & Wilkins, 1995).

Administration methods using the formulations described herein are within the scope of the invention. It is recommended that the person seeking appetite suppression follow the proceeding protocol to obtain maximal satiety. The capsule should be taken on an empty stomach (no solid food for 2 hours) to ensure rapid release of the capsule from the stomach. Approximately 45 minutes to one hour following ingestion of the capsule, at least one 8 ounce glass of liquid should be consumed. Filling or stretching the stomach with liquid will help further stimulate the afferent vagal fibers, which are already stimulated by the binding of CCK-8 to the CCK_{A} receptors. Along these lines, it may be helpful to drink a carbonated beverage such as seltzer water, which would cause more gastric distension than a non-carbonated beverage. Food may be consumed as desired after consumption of the liquid.

The formulations and methods of the invention may be administered prophylactically. The terms “controlling weight,” “weight control,” and permutations of these terms are used to encompass therapeutic as well as prophylactic uses. Hence, as used herein, by “controlling weight” is meant reducing, preventing, and/or reversing the weight gain of the individual to which a compound of the invention has been administered, as compared to the weight gain of an individual receiving no such administration.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLES

### Example 1

### Placebo-Controlled Study

This was a one month, double-blinded, placebo-controlled study measuring weight loss in 31 overweight patients (BMI of at least 25). Patients were instructed not to alter their diet, daily activities, or exercise habits during the study period. 15 patients received placebo, while 16 patients received enteric-coated capsules containing 2.4 micrograms of CCK-8. Patients were instructed to ingest one capsule, one hour before lunch and one hour before dinner. Prior to beginning each meal, patients were instructed to drink one 8 oz glass of water.

### Placebo

Mean weight loss was 0.67 lb. Percentage (mean) of the initial body weight lost was 0.29%.Median weight loss was 1.25 lb. Percentage (mean) of the initial body weight lost 0.0 lb.

### Active

Mean weight loss was 1.1 lb. Percentage (mean) of the initial body weight lost was 0.74%. Median weight loss was 2 lb. Percentage (mean) of the initial body weight lost was 1.05%.

### Example 2

### CCK With Enzyme Inhibitor

This was a four month non-placebo-controlled-study measuring weight loss in twenty overweight patients. Patients were instructed not to alter their diet, daily activities, or exercise habits during the study period. The patients were given enteric-coated-capsules containing 3.14 micrograms of CCK-8 and an intestinal enzyme inhibitor. Patients were allowed to take between one and three capsules before each meal. The capsules were ingested as in Study #1—one hour before lunch and one hour before dinner. Prior to beginning each meal, patients were instructed to drink one 8 oz glass of water.

Mean weight loss was 17 lb. over 4 months, while mean weight loss per month was 4.25 lb. The percentage (mean) of the initial body weight lost over 4 months was 8.6%. Maximum weight lost was 42 lb (this occurred in 2 patients). The maximum percentage of initial body weight lost was 21%, while minimum weight lost was 6 lb. Minimum percentage of initial body weight lost was 3.6% and the median weight lost was 11.5 lb over 4 months. Percentage (median) of initial body weight lost was 6.5%.

This study revealed significant weight loss, even though the doses of CCK-8 administered probably did not cause CCK-8 levels to rise into the range seen with ingestion of a fatty meal. The above weight loss data is consistent with the results seen with SIBUTRAMINE™ and XENICAL™, two drugs used for long-term treatment of obesity.

### Example 3

### Pharmacokinetics Study

The purpose of this study was to determine the peak concentration of CCK-8 achieved after three individuals each ingested one enteric-coated capsule, containing 9.2 micrograms CCK-8 and an enzyme inhibitor. Secondarily, it was desired to ascertain the time it would take to reach peak plasma levels. Each individual ingested the capsule in the fasting state (not having eaten for at least 8 hours). Blood was then drawn every 10 minutes for one hour. TRASYOL™ (which inhibits the degradation of CCK-8 in the blood) was added to each tube (per Mt. Sinai Hospital protocol, in Ontario, Canada) and the tubes were then centrifuged. Plasma was drawn off from each tube and frozen. The frozen plasma was sent to Mt. Sinai Hospital in Canada for measurement of CCK-8 levels. The following results were obtained:
Individual #1—At 10 minutes post-ingestion, a CCK-8 level of 3.268 picomoles/liter (pm/l) was measured. Each of the remaining 5 measurements (taken every 10 minutes) detected no CCK-8.
Individual #2—At 10 minutes post-ingestion, a CCK-8 level of 0.7625 pm/l was measured. At 20 minutes, a CCK-8 level of 3.326 pm/l was measured. The next 4 measurements detected no CCK-8.
Individual #3—At 10 minutes post-ingestion, a CCK-8 level of 0.0 pm/l was measured. At 20 minutes, a CCK-8 level of 4.375 pm/l was measured. The next 4 measurements detected no CCK-8.

### Discussion of Pharmacokinetics Study Results

CCK-8 levels in the fasting state are typically less than 1 pm/l. Following a meal containing fat, CCK-8 levels rise above 5.8 pm/l, and often are measured between 7 and 8 pm/l. As mentioned earlier, CCK-8 has never before been prepared in an orally bio-available fashion. Because oral bio-availability has been less than 1%, elevated CCK-8 levels had never been detected in response to an oral CCK-8 challenge.

The elevated CCK-8 levels measured above may be associated with some feelings of fullness but are not in the range of the levels seen after ingestion of a fatty meal. Individual #3 had the highest level; this most likely occurred because this individual had the lowest BMI (24), and therefore the ingested CCK-8 would have a smaller volume of distribution.

Based on the above results, it would be expected that a larger dose of CCK-8, administered in a capsule with enzyme inhibitor, would cause CCK-8 levels to rise into the range consistent with ingestion of a fatty meal.

### Example 4

### Representative Tablet Formulation According to the Invention

The following example illustrates one possible formulation according to the invention but should not be construed as a limitation thereto. In this instance, the appetite suppressing moiety was sulfated CCK-8 (Bachem A G, Bubendorf, Switzerland), the chelating agent CaEDTA, and the carrier was microcrystalline cellulose. The active ingredient, microcrystalline cellulose, and chelator were sieved through a 500 micron sieve and blended in a suitable mixer to form the active mix. Number 1 gelatin capsules (Hawkins Chemicals, Minneapolis, Minn.) were then filled with the active mix. Additional microcrystalline cellulose was added to the capsule to remove any remaining air space to inhibit oxidation of the active ingredient. Alternatively, the capsule may be filled under flowing nitrogen to remove any remaining free air from the capsule.

The filled capsules were then coated in a conventional manner with cellulose acetate phthalate solution prepared according to Table 1 below.

TABLE 1
Sample Enteric Coating Solution

| Ingredient | Amount | |
|---|---|---|
| Cellulose acetate phthalate | 11 | gm |
| Ammonium Hydroxide (27-31%) | 3.5 | ml |
| Ethyl alcohol (190 proof USP) | 66 | ml |
| Triacetin USP | 2.2 | ml |
| Methylene blue (1%) | 0.15 | ml |
| Purified water | 28 | ml |

All materials for the enteric coating solution were obtained from Spectrum Chemicals MFG Corp., (Tucson, Ariz.). The final coated capsules contained 4 μg of CCK8, 27 mg of microcrystalline cellulose NF, and 200 mg of USP grade CaEDTA.

### Example 5

### Weight Loss Effect of Representative Appetite Suppressing Moieties Administered Orally

The effects of cholecystokinin-8 were examined using a human model. Twenty overweight or obese men (BMI>26) were each orally administered (p.o.) an enterically coated appetite suppressing composition containing CCK-8 (4 μg) and CaEDTA (200 mg) or an identical looking placebo. They were then monitored every week over a 4 week period for weight loss. After the 4 week period, a greater than 35 fold increase in weight loss was observed wherein the treatment group showed a mean weight loss of approximately 17.8 lbs whereas the control group displayed only a 0.5 lb weight loss.

The results demonstrated that those individuals receiving the appetite suppressing composition have much greater weight reduction than those receiving placebo.

### Example 6

### Satiety Effect of Representative Appetite Suppressing Moieties Administered Orally

Forty human male subjects, 21 to 64 years of age, weighing 170 to 205 pounds, are selected. Half of the participants are orally administered the appetite suppressing composition in capsule form while the other half receive a placebo. The appetite suppressing composition contains 4 μg of CCK-8, 200 mg of CaEDTA, and an enteric coating. Each subject participates for 25 days and is asked to keep a daily journal of their appetite and meal size. The participants are weighed 5 days prior to taking the appetite suppressing composition and every 5 days while taking the appetite suppressing composition. Subjects are asked to take one capsule approximately 35 minutes before the largest meal of the day and not to eat any food 90 minutes prior to taking the capsule. Subjects are also asked to drink the capsule with a glass of water or seltzer water, and to drink another glass 30 minutes later. In addition subjects are asked to drink another glass of water or seltzer water with the meal. As postulated herein the addition of water or seltzer water allows for faster distension after taking the capsule thus allowing subjects to receive the maximum benefit from the capsule. After taking the capsule subjects are asked to write in their daily journals in 15 minute increments rating their satiety as follows:
0—Starving and beyond; so hungry that you are weak.
1—Too hungry; you feel like you could eat everything on the menu.
2—Very hungry; everything on the menu begins to look good, you may be preoccupied with hunger.
3—Moderately hungry; but not preoccupied with thoughts of hunger.
4—Mildly hungry; something light would suffice but you could wait another hour for the desire to develop more fully.
5—Satisfied; not hungry and not full.
6—A little fuller than satisfied; you could definitely eat more but the food is not as delicious.
7—Hunger is gone; your belly is not uncomfortable but you probably do not need another meal for another 3 to 4 hours.
8—Belly is full; and is mildly uncomfortable.
9—Belly is full and causing a moderate amount of discomfort.
10—Thanksgiving full; very uncomfortable.

The results should demonstrate that subjects taking the appetite suppressing composition will record greater satiety for a greater period than those on placebo.

### Example 7

### Caloric Intake Effect of Representative Appetite Suppressing Moieties Administered Orally

Each of twenty human male subjects, 21 to 64 years of age; weighing 170 to 205 pounds receive the same appetite suppressing composition as in Example 3. Another group of twenty human male subjects receive an appetite suppressing composition that contains 2.4 μg of CCK-8, 200 mg of CaEDTA, and an enteric coating. Subjects are asked to keep a daily journal of their caloric intake beginning 4 weeks prior to the administration of the appetite suppressing composition and ending 4 weeks after administration begins. The administration of the capsule is identical to Example 3. A control group of twenty human male subjects is administered placebo.

The results should indicate a reduction in caloric intake after the administration of the capsule than compared to before its administration. In addition, the results should show that the reduction in caloric intake is dose-dependent indicating that the appetite suppressing composition is not only useful to reduce caloric intake, but also to maintain a certain caloric intake. This result would be particularly useful in type II diabetes applications to control blood sugar and maintain lower weights than those associated with the adult onset of type II diabetes.

### Example 8

### Bioavailability of Orally Administered CCK-8

The bioavailability of-orally administered CCK-8 composition can be examined using a human model. Using 30 healthy subjects, 10 are orally administered an enterically coated appetite suppressing composition containing CCK-8 (4 μg) and CaEDTA (200 mg). Another 10 subjects are orally administered an enterically coated composition containing CCK-8 (4 μg). The third group of 10 subjects are orally administered a non-enterically coated composition containing CCK-8 (4 μg).

Bioavailability tests should demonstrate that those subjects taking the enterically coated appetite suppressing composition achieved a very high bioavailability, while those taking enterically coated cholecystokinin-8 alone had much lower bioavailability. In contrast, those taking non-enterically coated cholecystokinin-8 should have a bioavailability of about 0% because stomach peptidases should have destroyed most of the peptide before reaching the ileum.

What is claimed:

1. A pharmaceutical formulation for oral administration to a mammal to modulate satiety comprising an appetite suppressing peptide, and a chelating agent, wherein the pharmaceutical formulation is encased in an enteric coating or capsule.

2. A pharmaceutical formulation for oral administration to a mammal to reduce feeding comprising an appetite suppressing peptide, and a chelating agent, wherein the pharmaceutical formulation is encased in an enteric coating or capsule.

3. The pharmaceutical formulation of claim 1, further comprising one or more acceptable carriers.

4. The pharmaceutical formulation of claim 1, wherein said appetite suppressing peptide is either CCK or caerulein.

5. The pharmaceutical formulation of claim 4, where said CCK is selected from the group consisting of cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7 or cholecystokinin-4 (CCK-4). The pharmaceutical formulation of claim 2, further comprising one or more acceptable carriers.

6. The pharmaceutical composition of claim 1, wherein the chelating agent is selected from the group consisting of ethylenediamine-N,N,N′,N′-tetraacetic acid (EDTA); the disodium, trisodium, tetrasodium, dipotassium, tripotassium, dilithium and diammonium salts of EDTA; the barium, calcium, cobalt, copper, dysprosium, europium, iron, indium, lanthanum, magnesium, manganese, nickel, samarium, strontium, and zinc chelates of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-te-traacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephos-phoric acid); 7,19,30-trioxa-1,4,10,13,16,22,27,33-octaazabicyclo [11,11,11]pentatriacontane hexahydrobromide; and triethylenetetramine-N,-N,N′,N″,N′″,N′″-hexaacetic acid, citric acid, and phosphoric acid.

7. A method to modulate satiety comprising the step of orally administrating to a mammal a pharmaceutical formulation comprising an appetite suppressing peptide, and a chelating agent, wherein the formulation is encased in an enteric coating or capsule.

8. A method to reduce feeding comprising the step of orally administrating to a mammal a pharmaceutical formulation comprising an appetite suppressing peptide, and a chelating agent, wherein the formulation is encased in an enteric coating or capsule.

9. The method of claim 7, wherein the pharmaceutical formulation further comprises one or more acceptable carriers.

10. The method of claim 7, wherein said appetite suppressing peptide is either CCK or caerulein.

11. The method of claim 10, where said CCK is selected from the group consisting of cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7 or cholecystokinin-4 (CCK-4).

12. The method of claim 7, wherein the chelating agent is selected from the group consisting of ethylenediamine-N,N,N′,N′-tetraacetic acid (EDTA); the disodium, trisodium, tetrasodium, dipotassium, tripotassium, dilithium and diammonium salts of EDTA; the barium, calcium, cobalt, copper, dysprosium, europium, iron, indium, lanthanum, magnesium, manganese, nickel, samarium, strontium, and zinc chelates of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-te-traacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephos-phoric acid); 7,19,30-trioxa-1,4,10,13,16,22,27,33-octaazabicyclo [11,11,11]pentatriacontane hexahydrobromide; and triethylenetetramine-N,- N,N′,N″,N′″,N′″-hexaacetic acid, citric acid, and phosphoric acid.

13. The pharmaceutical formulation of claim 2, further comprising one or more acceptable carriers.

14. The pharmaceutical formulation of claim 2, wherein said appetite suppressing peptide is either CCK or caerulein.

15. The pharmaceutical formulation of claim 14, where said CCK is selected from the group consisting of cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7 or cholecystokinin-4 (CCK-4). The pharmaceutical formulation of claim 2, further comprising one or more acceptable carriers.

16. The pharmaceutical composition of claim 2, wherein the chelating agent is selected from the group consisting of ethylenediamine-N,N,N′,N′-tetraacetic acid (EDTA); the disodium, trisodium, tetrasodium, dipotassium, tripotassium, dilithium and diammonium salts of EDTA; the barium, calcium, cobalt, copper, dysprosium, europium, iron, indium, lanthanum, magnesium, manganese, nickel, samarium, strontium, and zinc chelates of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-te-traacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephos-phoric acid); 7,19,30-trioxa-1,4,10,13,16,22,27,33-octaazabicyclo [11,11,11]pentatriacontane hexahydrobromide; and triethylenetetramine-N,-N,N′,N″,N′″,N′″-hexaacetic acid, citric acid, and phosphoric acid.

17. The method of claim 8, wherein the pharmaceutical formulation further comprises one or more acceptable carriers.

18. The method of claim 8, wherein said appetite suppressing peptide is either CCK or caerulein.

19. The method of claim 18, where said CCK is selected from the group consisting of cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7 or cholecystokinin-4 (CCK-4).

20. The method of claim 8, wherein the chelating agent is selected from the group consisting of ethylenediamine-N,N,N′,N′-tetraacetic acid (EDTA); the disodium, trisodium, tetrasodium, dipotassium, tripotassium, dilithium and diammonium salts of EDTA; the barium, calcium, cobalt, copper, dysprosium, europium, iron, indium, lanthanum, magnesium, manganese, nickel, samarium, strontium, and zinc chelates of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-te-traacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephos-phoric acid); 7,19,30-trioxa-1,4,10,13,16,22,27,33-octaazabicyclo[11,11,11]pentatriacontane hexahydrobromide; and triethylenetetramine-N,-N,N′,N″,N′″,N′″-hexaacetic acid, citric acid, and phosphoric acid.

21. The pharmaceutical formulation of claim 2, wherein the pharmaceutical formulation is effective to increase satiety.

22. The method of claim 7 wherein the method results in an increase in satiety.

23. The method of claim 8, wherein the method results in a decrease in feeding.

24. The pharmaceutical composition of claim 6, wherein the chelating agent is citric acid.

25. The pharmaceutical composition of claim 16, wherein the chelating agent is citric acid.

26. The method of claim 12, wherein the chelating agent is citric acid.

27. The method of claim 20, wherein the chelating agent is citric acid.

28. The pharmaceutical formulation of claim 1, wherein the pharmaceutical formulation further comprises a lipid-based drug delivery system in the form of liposomes.

29. The pharmaceutical formulation of claim 2, wherein the pharmaceutical formulation further comprises a lipid-based drug delivery system in the form of liposomes.

30. The pharmaceutical formulation of claim 1, further comprising a bioactive substance operative to enhance the therapeutic effect of the appetite suppressing peptide.

31. The pharmaceutical formulation of claim 30, wherein the bioactive substance comprises at least one of:
an epinephrine antagonist, an opiate antagonist, a pancreatic polypeptide blocker, a GABA agonist, a serotonin agonist, a calcitonin agonist, a corticotrophin-releasing factor agonist, a neurotensin agonist, a dopamine agonist, an anaesthetic, a glucagons agonist, pro-opiomelanocortin, cocaine- and amphetamine-regulated transcript (CART), urotcortin, thyrotropin-releasing hormone, galanin-like peptide-1, peptide YY, ciliary neurotrophic factor, brain-derived neural factor, insulin, insulin-like growth factor-1, insulin-like growth factor-2, leptin, neuropeptide K, calcitonin-gene-related peptide, prolactin-releasing peptide, neuromedin, neuropeptide B, somatostatin, oxytocin, bombesin, motilin, enterostatin, anorectin, amylin, and interleukin 1.

32. The pharmaceutical formulation of claim 2, further comprising a bioactive substance operative to enhance the therapeutic effect of the appetite suppressing peptide.

33. The pharmaceutical formulation of claim 32, wherein the bioactive substance comprises at least one of:
an epinephrine antagonist, an opiate antagonist, a pancreatic polypeptide blocker, a GABA agonist, a serotonin agonist, a calcitonin agonist, a corticotrophin-releasing factor agonist, a neurotensin agonist, a dopamine agonist, an anaesthetic, a glucagons agonist, pro-opiomelanocortin, cocaine- and amphetamine-regulated transcript (CART), urotcortin, thyrotropin-releasing hormone, galanin-like peptide-1, peptide YY, ciliary neurotrophic factor, brain-derived neural factor, insulin, insulin-like growth factor-1, insulin-like growth factor-2, leptin, neuropeptide K, calcitonin-gene-related peptide, prolactin-releasing peptide, neuromedin, neuropeptide B, somatostatin, oxytocin, bombesin, motilin, enterostatin, anorectin, amylin, and interleukin 1.

34. The method of claim 7, wherein the pharmaceutical formulation further comprises a bioactive substance operative to enhance the therapeutic effect of the appetite suppressing peptide.

35. The method of claim 34, wherein the bioactive substance comprises at least one of:
an epinephrine antagonist, an opiate antagonist, a pancreatic polypeptide blocker, a GABA agonist, a serotonin agonist, a calcitonin agonist, a corticotrophin-releasing factor agonist, a neurotensin agonist, a dopamine agonist, an anaesthetic, a glucagons agonist, pro-opiomelanocortin, cocaine- and amphetamine-regulated transcript (CART), urotcortin, thyrotropin-releasing hormone, galanin-like peptide-1, peptide YY, ciliary neurotrophic factor, brain-derived neural factor, insulin, insulin-like growth factor-1, insulin-like growth factor-2, leptin, neuropeptide K, calcitonin-gene-related peptide, prolactin-releasing peptide, neuromedin, neuropeptide B, somatostatin, oxytocin, bombesin, motilin, enterostatin, anorectin, amylin, and interleukin 1.

36. The method of claim 8, wherein the pharmaceutical formulation further comprises a bioactive substance operative to enhance the therapeutic effect of the appetite suppressing peptide.

37. The method of claim 36, wherein the bioactive substance comprises at least one of:
an epinephrine antagonist, an opiate antagonist, a pancreatic polypeptide blocker, a GABA agonist, a serotonin agonist, a calcitonin agonist, a corticotrophin-releasing factor agonist, a neurotensin agonist, a dopamine agonist, an anaesthetic, a glucagons agonist, pro-opiomelanocortin, cocaine- and amphetamine-regulated transcript (CART), urotcortin, thyrotropin-releasing hormone, galanin-like peptide-1, peptide YY, ciliary neurotrophic factor, brain-derived neural factor, insulin, insulin-like growth factor-1, insulin-like growth factor-2, leptin, neuropeptide K, calcitonin-gene-related peptide, prolactin-releasing peptide, neuromedin, neuropeptide B, somatostatin, oxytocin, bombesin, motilin, enterostatin, anorectin, amylin, and interleukin 1.
